SEQUENCE LISTING (14 sequences)

SEQ ID NO: 1 - DNA, 24 bp, Artificial
  oligonucleotide
tgcagccaaagtgaagagggaaga

SEQ ID NO: 2 - DNA, 24 bp, Artificial
  oligonucleotide
catagcgagcagcccaaagaagaa

SEQ ID NO: 3 - DNA, 24 bp, Artificial
  oligonucleotide
tgcttgaatgtgctgatgacaggg

SEQ ID NO: 4 - DNA, 25 bp, Artificial
  oligonucleotide
aaggcaagtcagcaggcatctcatc

SEQ ID NO: 5 - DNA, 22 bp, Artificial
  oligonucleotide
tggtactctcctcaatctgctg

SEQ ID NO: 6 - DNA, 21 bp, Artificial
  oligonucleotide
ctctggattgactgtggaagt

SEQ ID NO: 7 - DNA, 21 bp, Artificial
  oligonucleotide
tgagcagtctgtccactgcct

SEQ ID NO: 8 - DNA, 22 bp, Artificial
  oligonucleotide
atgtgaatgaggaggatctgag

SEQ ID NO: 9 - DNA, 20 bp, Artificial
  oligonucleotide
atacagagacttcagggagc

SEQ ID NO: 10 - DNA, 23 bp, Artificial
  oligonucleotide
tggttgggttcatcttcggtatc

SEQ ID NO: 11 - DNA, 23 bp, Artificial
  oligonucleotide
gctggagtcctgtcaggcattgc

SEQ ID NO: 12 - DNA, 22 bp, Artificial
  oligonucleotide
tagagctgaggcggaatgggag

SEQ ID NO: 13 - DNA, 20 bp, Artificial
  oligonucleotide
tgaactggctgactgctgtg

SEQ ID NO: 14 - DNA, 20 bp, Artificial
  primer
catccttggcctcagcatag

(19) United States
(12) Patent Application Publication
(10) Pub. No.: US 2005/0233449 A1
(43) Pub. Date: Oct. 20, 2005

(54) Simple and rapid derivation of functional hepatocytes from human bone marrow-derived mesenchymal stem cells

(76) Inventors: Kuan-Der Lee, Chiayi City (TW); Jacqueline Whang-Peng, Taipei City (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/099,451

(22) Filed: Apr. 6, 2005

Related U.S. Application Data
(60) Provisional application No. 60/563,194, filed on Apr. 16, 2004.

(51) Int. Cl. C12N 5/08

(52) U.S. Cl. 435/370

(57) ABSTRACT

This disclosure provides methods for preparing hepatocytes from mesenchymal stem cells by culturing in a first culture media comprising hepatocyte growth factor and a second culture media comprising oncostatin-M. The disclosure also provides the MSC-derived hepatocytes produced by these methods and both in vivo and in vitro uses for these MSC-derived hepatocytes.

This application claims the benefit of U.S. provisional application No. 60/563,194, filed Apr. 16, 2004, which is incorporated herein by reference.

### DESCRIPTION OF THE INVENTION

1. Field of the Invention

The invention relates to the use of mesenchymal stem cells (MSCs) for the in vitro production of hepatocytes and hepatic tissues. This invention further relates to the use of MSC-derived hepatocytes in methods to evaluate and treat liver diseases.

2. Background of the Invention

The increasing incidence of severe liver diseases coupled with a continual shortage of donor organs for orthotopic liver transplantation highlights the need for alternative strategies for treating liver diseases. One potential therapy involves the use of extracorporeal bioartificial liver (Allen et al., Tissue Eng 2002, 8:725-37), where isolated hepatocytes are integrated with membrane-based bioreactors. In addition, in vitro tissue culture models of parenchymal liver cells are of great importance in viral hepatitis and toxicological research.

There are currently three sources for cultured hepatocytes: primary human cultures, cultured animal hepatocytes, and transformed hepatocyte cell lines. A shortage of liver donors, low residual function after cryopreservation, and loss of most liver functions after primary culture makes the use of primary cultures of hepatocytes difficult. (Klocke et al., Biochem Biophys Res Commun 2002, 294:864-71). The use of cultured animal hepatocytes raises important concerns regarding the possible transmission of a xenozoootic agent, such as porcine retrovirus. The use of transformed hepatoblastoma cell lines is hampered by insufficient liver function and the risk of transmigration of tumorigenic cells into the body. Thus, use of any of the currently available hepatic cells in clinical applications is not practical. Therefore, alternative sources of hepatocytes are needed.

Stem cells responsible for self-repair and regeneration are found in various organs of the human body. However, human hepatic stem cells (oval cells) have remained illusive and the mechanism responsible for the regenerative capacity of liver tissue is controversial (Suzuki et al., Hepatology 2000, 32:1230-1239). An extrahepatic source of stem or progenitor cells for the derivation of hepatocytes would provide a much needed supply of functional hepatocytes.

Recent studies have demonstrated that adult stem cells are capable of differentiating into cells different from their germ layer of origin (Woodbury et al., J Neurosci Res 2000, 61:364-370). Adult bone marrow is a reservoir of various stem cells including hematopoietic stem cells (HSCs), mesenchymal stem cells (MSCs), and multipotent adult progenitor cells (MAPCs). MAPCs are the only stem cells known to differentiate into tissue of all three germ lines (mesodermal, ectodermal, and endodermal) (Schwartz et al., J Clin Invest 2002, 109:1291-1302). However, MAPCs are very rare, and it is not known whether these cells are normally present in bone marrow or if they are an artifact of in vitro culture (Herzog et al., Blood 2003, 102:3483-3493; Reyes et al., Ann N Y Acad Sci 2001, 938:231-235). Therefore, MAPCs are not a reliable or easily obtainable source of stem cells for hepatic differentiation.

There has been a suggestion that bone marrow may be a source of hepatic-determined stem cells in vivo (Theise et al., Semin Cell Dev Biol 2002, 13:411-417; Petersen et al., Science 1999, 284:1168-1170; Lagasse et al., Nat Med 2000, 6:1229-1234). However, the results reported in these references are controversial. More recent reports demonstrate that, rather than differentiating into hepatocytes, the bone marrow-derived stem cells fuse with hepatic cells to change cell type. (Wang et al., Nature 2003, 422:897-901; Vassilopoulos et al., Nature 2003, 422:901-904). This cell fusion phenomenon has been shown to result in the production of not only hepatocytes, but chondrocytes, cardiac cells, and others (Terada et al., Nature 2002, 416:542-545). The in vitro production of hepatocytes from stem cells has not been demonstrated.

MSCs, widely studied over the past three decades and readily accessible from bone marrow, have been shown to differentiate into mesodermal and ectodermal tissue in vitro. However, there has been no evidence prior to this invention that MSCs are capable of differentiating into endodermal tissue, such as liver or pancreas, in vitro. With this invention, the in vitro differentiation of MSCs into endodermal tissue has been achieved for the first time, making it possible to provide a simple, rapid in vitro hepatic differentiation system for marrow derived MSCs.

### SUMMARY OF THE INVENTION

The invention is based, in part, on the discovery that mesenchymal stem cells are capable of differentiating into hepatocytes under certain culture conditions. Accordingly, the invention provides methods for inducing differentiation of mesenchymal stem cells into hepatocytes in vitro. Embodiments of these methods comprise incubating cultured mesenchymal stem cells with a first culture media comprising hepatocyte growth factor (HGF) followed by incubation with a second culture media comprising oncostatin-M (OSM).

The MSCs may be of mammalian origin, and in particular embodiments, may be of human origin. In certain embodiments, prior to culturing, the MSCs may be subjected to immunodepletion of cells expressing CD3, CD14, CD19, CD38, CD66b, and/or glycophorin A.

In some embodiments, the first culture media further comprises fibroblast growth factor, nicotinamide, or both. In certain embodiments, the second culture media further comprises dexamethasone, insulin, or both.

The invention further provides methods for using MSC-derived hepatocytes to repair liver damage in a patient or for growing liver tissue in vitro. In particular embodiments, the MSC-derived hepatocytes are used to create a bioartificial liver device.

Further embodiments include the MSC-derived hepatocytes of the invention and/or liver tissue produced from the MSC-derived hepatocytes. The MSC-derived hepatocytes or liver tissue of the invention may be employed in methods of screening a compound for its effect on hepatocytes or a hepatocyte activity.

In some embodiments, the screening method is used to determine whether the compound is toxic to hepatic cells in the population. In other embodiments, the screening method is used to determine whether the compound affects the ability of hepatic cells in the population to proliferate or be maintained in culture.

In other embodiments, the MSC-derived hepatocytes are used to culture and grow liver cell-specific microbes, such as hepatitis viruses.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIGS. 1A-1J show the characterization and mesodermal differentiation of freshly isolated and long-term cryopreserved bone marrow-derived MSCs. FIG. 1A shows the morphology of MSCs at lower confluence. FIG. 1B shows the morphology at higher confluence. FIG. 1C shows flow cytometry results, demonstrating that these MSCs are negative for CD34, CD45, and CD133, but positive for CD29, CD71, CD73, CD90, and CD105 expression. In FIGS. 1D-F, MSCs are induced to differentiate into osteocyte-like cells (1D) and stain positive for alkaline phosphatase (1E) and mineralized matrices by Von Kossa assay (1F). In FIGS. 1G-H, MSCs are subjected to adipogenic conditions. These cells accumulate neutral lipid vacuoles (1G) that are positively stained by Oil red-O assay (1H). In FIGS. 1I-J, MSCs are subjected to chondrogenic conditions. These cells differentiate into chrondrocyte-like cells that stain positively for sulfated proteoglycans by safranin-O (1I) and for type II collagen by immunohistochemical analysis (1J).

FIGS. 2A-H show the in vitro hepatic differentiation of marrow-derived MSCs. FIGS. 2A-F show the morphology of the MSCs at the following timepoints: (2A) Undifferentiated MSCs; (2B) 1 week post induction; (2C) 2 weeks post induction; (2D) 4 weeks post induction; (2E) 6 weeks post induction; and (2F) 12 weeks post induction. FIGS. 2G-H show the expression of hepatocyte-specific marker genes by RT-PCR at the indicated time points (2G) and staining for albumin by immunofluorescence analysis (2H).

FIGS. 3A-J show the functional characterization of MSC-derived hepatocytes. FIG. 3A shows LDL uptake. FIG. 38 shows cytochrome P450 enzyme activity, as analyzed by PROD assay. FIG. 3C shows the induction of cytochrome P450 enzyme activity in the presence of phenobarbital. FIG. 3D shows an absence of glycogen storage in undifferentiated MSCs with a PAS assay. FIG. 3E shows the presence of glycogen storage in MSC-derived hepatocytes with a PAS assay. FIG. 3F shows that glycogen storage disappears when the MSC-derived hepatocytes are pre-treated with diastase to remove glycogen. FIG. 3G shows a time course of urea secretion of MSC-derived hepatocytes into culture medium as measured by urea assay. FIG. 3H shows that MSC-derived hepatocytes express bile canaliculi-specific antigen 9B2 as determined by flow cytometry. FIG. 3I shows immunofluorescent staining revealing that the antigen 9B2 is predominantly localized on the junction between adjacent MSC-derived hepatocytes. FIG. 3J shows immunofluorescence analysis on Hep3B cell line revealing a similar 9B2 staining pattern.

FIGS. 4A-F show the culture re-expansion of MSC-derived hepatocytes. FIG. 4A shows the hepatocytes re-expanded after 6 weeks of induction. FIG. 4B demonstrates that the cuboidal morphology of hepatocytes is lost while cells proliferate in the re-expansion medium. FIG. 4C shows that the cuboidal morphology is re-established after changing to the second medium containing OSM. FIG. 4D shows the cells to be further matured once cultures reach 80-90% confluence. FIG. 4E shows confirmation of the proliferation of the differentiated cells in re-expansion medium by tritium-thymidine (³H) incorporation analysis. FIG. 4F shows that the re-expanded hepatocyte-like cells retain the function of low-density lipoproteins uptake.

### DESCRIPTION OF THE EMBODIMENTS

Definitions

In order for the present invention to be more readily understood, certain terms are defined herein. Additional definitions are set forth throughout the detailed description.

The terms “hepatocyte,” “hepatic cell,” “liver cell,” and their cognates refer to cells derived from or present in a liver or liver tissue, or cells phenotypically similar to cells derived from a liver or liver tissue. The cells may be similar to cells derived from or present in the liver or liver tissue of any animal.

The terms “hepatocyte growth factor,” “HGF,” and their cognates refer to members of the family of cytokines described, for example, in Nakamura et al., Nature 1989, 342:440-43, as well as homologs of these cytokines from other species and naturally-occurring allelic variants of these proteins. The terms also refer to mutated versions of HGF that maintain the ability to induce the growth and differentiation of mesenchymal stem cells into hepatocytes.

The terms “fibroblast growth factor,” “FGF,” and their cognates refer to the family of cytokines described, for example, in Kurokawa et al., FEBS Lett 1987, 213(1):189-94, as well as homologs of these cytokines from other species and naturally-occurring allelic variants of these proteins. Any protein characterized as a fibroblast growth factor will be encompassed by these terms, including, but not limited to, FGF-1, FGF-2, FGF-3, and FGF-4.

The term “stem cell” refers to any cell with the ability to differentiate into another type of cell.

The term “endodermal cell” and its cognates refers to any cell of endodermal origin, including, but not limited to hepatocytes, pancreatic cells, urinary bladder cells, and cells producing the epithelium of the GI and respiratory tracts.

The terms “mesenchymal stem cell” and “MSC” refer to stem cells derived from adult bone marrow. Mesenchymal stem cells are also known as bone marrow stromal stem cells.

The term “MSC-derived hepatocytes” refers to the hepatocytes produced by the methods described herein, and specifically, to hepatocytes resulting from directed differentiation of MSCs that have been incubated with a first culture media comprising hepatocyte growth factor and subsequently incubated with a second culture media comprising oncostatin-M.

The term “test compound” refers to any compound or composition, chemical or biological, to be evaluated in the methods of the invention.

The terms “therapeutic compound” and “therapeutic,” used herein, refer to any compound capable of treating, reversing, ameliorating, halting, slowing progression of, or preventing clinical manifestations of a disorder, or of producing a desired biological outcome.

The terms “culture media,” “tissue culture media,” “incubation media,” “media,” and their cognates refer to solutions used for the nutritional and growth needs of cells grown in vitro. The terms “first incubation media” and “differentiation media” refer to media comprising HGF. The terms “second incubation media” or “maturation media” refer to media comprising OSM. The term “re-expansion media” refers to media comprising HGF and/or OSM.

The term “hepatic activity” refers to any biological activity normally present in liver cells or tissues.

Additional definitions of these and other terms will be provided throughout the specification.

The invention, is based, in part, on the discovery that MSCs can differentiate into hepatocytes upon treatment with a series of growth and differentiation factors.

Certain embodiments of the invention are based, in part, on the discovery that incubation of MSCs in the presence of hepatocyte growth factor followed by incubation in the presence of oncostatin-M induced the differentiation of the MSCs into hepatocytes. Thus, methods for inducing MSCs to grow and differentiate into hepatocytes comprise incubating the mesenchymal stem cells in the presence of hepatocyte growth factor followed by incubation in the presence of oncostatin-M.

Prior to carrying out the methods of making MSC-derived hepatocytes according to the invention, cultured MSCs may be prepared by any suitable protocol. One such protocol includes, e.g., the following steps:

(1) Bone marrow-derived MSCs are isolated from a patient donor or other suitable source. The donor may be the same patient that the MSC-derived hepatocytes may eventually be transplanted into, or it may be an autologous donor. In some embodiments, the MSCs are harvested from the iliac crest of the donor.

(2) Once the MSCs are isolated, they may optionally be subjected to immunodepletion of at least CD3, CD14, CD19, CD38, CD66b, and/or glycophorin A-positive cells. The MSCs remaining after immunodepletion are seeded into adherent tissue culture flasks and grown to about 50-60% confluency.

(3) The cultured MSCs may then be serum deprived for an amount of time sufficient to synchronize their cell cycles. This synchronization step may last from 0 days to about 5 days, about 1 day to about 4 days, or about 2 days.

In accordance with the methods of the invention, cultured MSCs may then be first incubated with a first culture media containing hepatocyte growth factor (HGF). This first media may optionally contain other compounds that provide additive effects, such as FGF and nicotinamide. These additive compounds improve the hepatic morphology of the cells, but do not affect gene or protein expression. The MSCs are incubated in the first media for a suitable time, usually between 5 and 21 days.

After the first incubation, the HGF-containing media is removed and a second culture media comprising oncostatin-M (OSM) is introduced. The second incubation is carried out for a suitable period, usually between 4 and 12 weeks. During this second incubation, OSM induces the hepatocytic phenotype in the MSCs and induces the expression of hepatocyte-specific genes. The second media may optionally contain other compounds that provide additive effects, such as dexamethasone and insulin. These additive compounds improve the hepatic morphology of the cells, but do not affect gene or protein expression.

After the second incubation with OSM, the resulting MSC-derived hepatocytes can be continually cultured and expanded in the presence of HGF and/or OSM. These MSC-derived hepatocytes will maintain the hepatocytic phenotype for up to 12 weeks.

In certain embodiments, the MSCs are first incubated with HGF in a concentration of at least 5 ng/ml. In other embodiments, the concentration of HGF is at least 10 ng/ml, at least 20 ng/ml, at least 50 ng/ml, at least 100 ng/ml, at least 500 ng/ml, or at least 1000 ng/ml. In specific embodiments, the concentration of HGF is about 10 ng/ml to about 50 ng/ml, about 15 ng/ml to about 25 ng/ml, or about 20 ng/ml.

In some embodiments, OSM is present in the second media in a concentration of at least 5 ng/ml. In other embodiments, the concentration of OSM is at least 10 ng/ml, at least 20 ng/ml, at least 50 ng/ml, at least 100 ng/ml, at least 500 ng/ml, or at least 1000 ng/ml. In particular embodiments, the concentration of OSM is about 10 ng/ml to about 50 ng/ml, about 15 ng/ml to about 25 ng/ml, or about 20 ng/ml.

In certain embodiments, the first culture media comprises fibroblast growth factor (FGF) in addition to HGF. In some embodiments, the FGF is FGF-1, FGF-2, FGF-3, and/or FGF-4. In particular embodiments, the FGF is FGF-2 and/or FGF-4. In further embodiments, the concentration of FGF in the first culture media is at least 1 ng/ml, at least 5 ng/ml, at least 10 ng/ml, at least 25 ng/ml, or at least 40 ng/ml. In particular embodiments, the concentration of FGF is about 1 to about 20 ng/ml, about 5 to about 15 ng/ml, or about 10 ng/ml.

In certain embodiments, the first culture media comprises nicotinamide in addition to HGF. Generally, the nicotinamide is supplied at typical levels for tissue culture methods. In particular embodiments, about 0.01 g/ml to about 0.60 g/ml of nicotinamide is added to the first culture media. In some embodiments, both FGF and nicotinamide are included in the first culture media with HGF.

In certain embodiments, the second culture media comprises insulin in addition to OSM. Generally, insulin is added to the second culture media at typical levels for tissue culture methods. In some embodiments, the insulin concentration in the second culture media is about 0.01% to about 3.0%. In particular embodiments, the insulin concentration in the second media is 1%.

In certain embodiments, the second culture media comprises dexamethasone in addition to OSM. In particular embodiments, the concentration of dexamethasone in the second culture media may be at least 0.1 μM, at least 0.5 μM, at least 1 μM, at least 5 μM, or at least 10 μM. In certain embodiments, the concentration of dexamethasone may be about 0.1 μM to about 10 μM, about 0.5 μM to about 5 μM, about 1<μM to about 2 μM, or about 1 μM. In some embodiments, both insulin and dexamethasone are added to the second culture media.

In some embodiments, MSCs are incubated with the first media comprising HGF for at least 5 days, at least 6 days, at least 7 days, at least 10 days, at least 14 days, or at least 21 days. In particular embodiments, the MSCs are incubated with the first media comprising HGF for about 5 to about 21 days, about 6 to about 14 days, about 7 to about 10 days, about 7 days, about 10 days, or about 14 days.

In some embodiments, the second incubation carried out in media comprising OSM continues for at least 4 weeks, at least 5 weeks, at least 8 weeks, at least 10 weeks, or at least 12 weeks. In particular embodiments, the second incubation continues for about 4 to about 12 weeks, about 6 to about 10 weeks, about 4 weeks, or about 8 weeks.

The invention also provides MSC-derived hepatocytes and compositions comprising MSC-derived hepatocytes. In some embodiments the MSC-derived hepatocytes are freshly differentiated, while in other embodiments, the MSC-derived hepatocytes have been differentiated accordingly to the methods of the invention, frozen down as cell stocks, and recultured at a later time. The MSC-derived hepatocytes of the invention are characterized by a cuboidal morphology, granules in the cytoplasm, and/or the expression of one or more hepatocyte-specific genes, such as, e.g., a-fetoprotein, glucose-6-phosphatase, and tyrosine-aminotransferase.

Other embodiments of the invention include liver and liver-like tissue comprising MSC-derived hepatocytes, lysates of MSC-derived hepatocytes, and conditioned media produced by culturing MSC-derived hepatocytes.

Additional embodiments of the invention include kits for producing MSC-derived hepatocytes. These kits may include cultured and optionally immunodepleted MSCs, a first incubation media comprising HGF, a second incubation media comprising OSM, and the appropriate protocols for producing the MSC-derived hepatocytes. In alternative embodiments, the kits would not include cultured MSCs, but would contain protocols for isolating MSCs from a patient and optionally antibodies for performing immunodepletions.

Another aspect of the invention provides methods for in vitro research on hepatocytes using the methods and compositions of the invention. In some embodiments, the MSC-derived hepatocytes are used to study liver cell specific viruses. In other embodiments, the MSC-derived hepatocytes are used to establish in vitro models of liver damage. In further embodiments, the MSC-derived hepatocytes are used to measure the expression of liver-specific genes. In additional embodiments, the MSC-derived hepatocytes are used to determine the effects of test compounds on liver tissue in vitro.

The MSC-derived hepatocytes of the invention can also be used to prepare a cDNA library relatively uncontaminated with cDNA preferentially expressed in cells from other lineages. Methods for preparing cDNA are well known in the art. The resulting cDNA can be subtracted with cDNA from any or all of the following cell types: undifferentiated MSCs, embryonic fibroblasts, visceral endoderm, sinusoidal endothelial cells, bile duct epithelium, or other cells of undesired specificity, thereby producing a select cDNA library, reflecting expression patterns that are representative of mature hepatocytes.

The MSC-derived hepatocytes of this invention can also be used to prepare antibodies that are specific for hepatocyte markers and other antigens that may be expressed on the cells. The MSC-derived hepatocytes provide an improved way of raising such antibodies because they are relatively enriched for particular cell types compared with MSC cell cultures and hepatocyte cultures made from liver tissue. Methods for preparing polyclonal antibodies, monoclonal antibodies, and antibody fragments are well known in the art. The antibodies in turn can be used to identify or rescue hepatocyte precursor cells of a desired phenotype from a mixed cell population, for purposes such as co-staining during immunodiagnosis using tissue samples, and isolating such cells from mature hepatocytes or cells of other lineages.

The MSC-derived hepatocytes of the invention are useful identifying the gene expression profile of hepatocytes at both the RNA and protein levels. Gene expression patterns of the MSC-derived hepatocytes are obtained and compared with control cell lines, such as undifferentiated MSCs cells, other types of committed precursor cells (such as MSCs differentiated towards other lineages), hematopoietic stem cells, precursor cells for other mesoderm-derived tissue, or precursor cells for endothelium or bile duct epithelium. Genes will be considered relevant to the gene expression profile if, when compared to control cell lines, their relative expression level is at least about 2-fold, 10-fold, or 100-fold elevated or suppressed in the MSC-derived hepatocytes of this invention.

Suitable methods for analyzing gene expression profiles at the protein level include immunoassay, mass spectrometry, or immunohistochemistry techniques known in the art. Suitable methods for analyzing gene expression profiles at the RNA level include methods of differential display of mRNA and microarray expression. These methods are well known in the art, and systems and reagents for performing these analyses are commercially available from a number of companies.

MSC-derived hepatocytes may be used in screening methods to identify compounds suitable for further drug research. In this invention, MSC-derived hepatocytes play the role of test cells for standard drug screening and toxicity assays, as have been previously performed on hepatocyte cell lines or primary hepatocytes in short-term culture. Assessment of the activity of candidate pharmaceutical compounds generally involves combining the differentiated cells of this invention with the candidate compound, determining any change in the morphology, marker phenotype, or metabolic activity of the cells that is attributable to the compound (compared with untreated cells or cells treated with an inert compound), and then correlating the effect of the compound with the observed change. The particular test compound may be selected (1) to determine whether the test compound has any pharmacological effect on liver cells; (2) to confirm that a test compound designed to have a pharmacological effect on liver cells actually does have that effect; or (3) to evaluate whether a compound designed to have effects elsewhere may have unintended site effects on hepatic cells and tissue. Two or more drugs can be tested in combination (by combining with the cells either simultaneously or sequentially), to detect possible drug-drug interaction effects. In some methods, compounds are screened initially for potential hepatotoxicity. Methods and assays for evaluating test compounds and measuring their effect on liver cells, including hepatoxicity, are well known in the art and are described in U.S. Pat. No. 6,506,574, which is hereby incorporated by reference.

This invention also provides for the use of MSC-derived hepatocytes to restore a degree of liver function to a subject needing such therapy, perhaps due to an acute, chronic, or inherited impairment of liver function.

To determine the suitability of differentiated hepatocytes for therapeutic applications, the MSC-derived hepatocytes can first be tested in a suitable animal model. MSC-derived hepatocytes may be assessed for their ability to survive and maintain their phenotype in vivo by administering them to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether MSC-derived hepatocytes are still present. This can be performed by providing the administered cells with a detectable label (such as green fluorescent protein or β-galactosidase); or by measuring a constitutive marker specific for the administered cells. Where human. MSC-derived hepatocytes are being tested in a rodent model, the presence and phenotype of the administered human MSC-derived hepatocytes can be assessed by immunohistochemistry or ELISA using human-specific antibody, or by RT-PCR analysis using primers and hybridization conditions that cause amplification to be specific for human polynucleotide sequences. General descriptions for determining the fate of human cells in animal models are provided in Grompe et al., Sem Liver Dis 1999, 19:7; Peeters et al., Hepatology 1997, 25:884; and Ohashi et al., Nature Med 2000, 6:327.

The differentiated hepatocytes may also be assessed for their ability to restore liver function in an animal lacking full liver function. Braun et al., Nature Med 2000, 6:320; Rhim et al., Proc Natl Aced. Sci USA 1995, 92:4942; Lieber et al., Pro Natl Acad Sci USA 1995, 92:6210; Overturf et al., Human Gene Ther 1998, 9:295; and Mignon et al., Nature Med 1998, 4:1185, describe various animal models for liver disease. Acute liver disease can be modeled by 90% hepatectomy. (See, Kobayashi et al., Science 2000, 287:1258). Acute liver disease can also be modeled by treating animals with a hepatotoxin such as galactosamine, CCl₄, or thioacetamide. Chronic liver diseases such as cirrhosis can be modeled by treating animals with a sub-lethal dose of a hepatotoxin long enough to induce fibrosis. (Rudolph et al., Science 2000, 287:1253).

Assessing the ability of MSC-derived hepatocytes to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, blood indicators (such as alkaline phosphatase activity, bilirubin conjugation, and prothrombin time), and survival of the host. Any improvement in survival, disease progression, or maintenance of hepatic function according to any of these criteria relates to effectiveness of the therapy, and can lead to further optimization.

The invention includes MSC-derived hepatocytes that are encapsulated in a bioartificial liver device. The success of a bioartificial liver device relies on a safe, readily available source of hepatocyte cells. The MSC-derived hepatocytes of this invention can be expanded to a large scale satisfying the need of critical cell number for bioartificial liver devices (i.e., 10¹¹ cells). Various forms of encapsulation of hepatocytes in bioartificial liver devices are described in Kuhtreiber et al. eds., Cell Encapsulation Technology and Therapeutics 1999, Birkhauser, Boston, Mass. Differentiated cells of this invention can be encapsulated according to such methods for use either in vitro or in vivo.

Bioartificial organs for clinical use are designed to support an individual with impaired liver function—either as a part of long-term therapy, or to bridge the time between a fulminant hepatic failure and hepatic reconstitution or liver transplant. Bioartificial liver devices are described in U.S. Pat. Nos. 5;290,684; 5,624,840; 5,837,234; 5,853,717; and 5,935,849. Suspension-type bioartificial livers comprise hepatic cells suspended in plate dialyzers, or microencapsulated in a suitable substrate, or attached to microcarrier beads coated with extracellular matrix. Alternatively, the hepatocytes can be placed on a solid support in a packed bed, in a multiplate flat bed, on a microchannel screen, or surrounding hollow fiber capillaries.

These devices may comprise preparative cultures of human cells that perform liver functions in vitro. The MSC-derived hepatocytes can be plated into the in vitro device on a suitable substrate, such as a matrix of Matrigel® or collagen. The device has inlet and outlet ports through which the subject's blood is passed, and sometimes a separate set of ports for supplying nutrients to the cells. The efficacy of the device can be assessed by comparing the composition of blood in the afferent channel with that in the efferent channel—in terms of metabolites removed from the afferent flow, and newly synthesized proteins in the efferent flow.

Devices of this kind can be used to detoxify a fluid such as blood, wherein the fluid comes into contact with the MSC-derived hepatocytes of this invention under conditions that permit the cell to remove or modify a toxin in the fluid. In the context of therapeutic care, the device processes blood flowing from a patient with liver damage, and then the blood is returned to the patient.

MSC-derived hepatocytes that demonstrate desirable functional characteristics in animal models may also be suitable for direct administration to human subjects with impaired liver function. For purposes of hemostasis, the MSC-derived hepatocytes, alone or in devices, can be administered at any site that has adequate access to the circulation, typically within the abdominal cavity. For some metabolic and detoxification functions, it is advantageous for the MSC-derived hepatocytes to have access to the biliary tract.

The MSC-derived hepatocytes can be used for therapy of any subject needing hepatic function restored or supplemented. Human conditions that may be appropriate for such therapy include fulminant hepatic failure due to any cause, viral hepatitis, drug-induced liver injury, cirrhosis, inherited hepatic insufficiency (such as Wilson's disease, Gilbert's syndrome, or α-antitrypsin deficiency), hepatobiliary carcinoma, autoimmune liver disease (such as autoimmune chronic hepatitis or primary biliary cirrhosis), liver damage as a result of chemotherapy designed to kill cancerous liver cells, and any other condition that results in impaired hepatic function. For human therapy, the dose is generally about 10⁹ to 10¹² cells, and typically about 5×10⁹ to 5×10¹⁰ cells, making adjustments for the body weight of the subject, nature and severity of the affliction, and the replicative capacity of the administered MSC-derived hepatocytes.

Genetically-altered MSC-derived hepatocytes may also be used in the devices and therapies described above. MSC-derived hepatocytes may be transfected or transformed with a recombinant gene in vitro, either before or after differentiation The genetically-altered MSC-derived hepatocytes may be administered to a patient as described above to produce a desired recombinant therapeutic protein in vivo, leading to clinical improvement of the patient. For example, treatment of affected patients with MSC-derived hepatocytes genetically-altered to produce recombinant clotting factors avoids the potential risk of exposing patients to viral contaminants, such as viral hepatitis and human immunodeficiency virus.

Conventional gene transfer methods may be used to introduce DNA into MSC-derived hepatocytes. The precise method used to introduce a replacement gene (e.g., one encoding a clotting factor or metabolic protein) is not critical to the invention. For example, physical methods for the introduction of DNA into cells include microinjection and electroporation. Chemical methods such as co-precipitation with calcium phosphate and incorporation of DNA into liposomes are also standard methods of introducing DNA into mammalian cells. DNA may be introduced using standard vectors, such as those derived from murine and avian retroviruses. See, e.g., Gluzman et al., Viral Vectors 1988, Cold Spring Harbor Laboratory, Cold Spring Harbor, N.Y. Standard recombinant DNA methods are well known in the art (Ausubel et al., Current Protocols in Molecular Biology 1989, John Wiley & Sons, New York) and viral vectors for gene therapy have been developed and successfully used clinically (Rosenberg et al., N Engl J Med 1990, 323:370).

It is to be understood that both the foregoing general description and the following detailed description are exemplary and explanatory only and are not restrictive of the invention, as claimed.

The accompanying examples, which are incorporated in and constitute a part of this specification, illustrate several embodiments of the invention and together with the description, serve to explain the principles of the invention.

### EXAMPLE 1

### MSC Isolation and Culture

Cytokines: Basic-fibroblast growth factor (bFGF), hepatocyte growth factor (HGF), and oncostatin M (OSM) were purchased from R&D Systems (Minneapolis, Minn.).

Isolation: Human bone marrow was aspirated from the iliac crest with informed consent. Mononuclear cells were obtained by negative immunodepletion of CD3, CD14, CD19, CD38, CD66b, and glycophorin-A positive cells using a commercially available kit (RosetteSep®, StemCell Technologies, Vancouver, BC, Canada), followed by Ficoll-Paque® (Amersham-Pharmacia, Piscataway, N.J., USA) density gradient centrifugation (1.077 g/cm³). The MSCs were then plated in non-coated tissue culture flasks (Becton Dickinson) in expansion medium (Iscove's modified Dulbecco's medium (IMDM, Gibco BRL, Grand Island, N.Y.) and 10% Fetal Bovine Serum (FBS, Hyclone, Logan, Utah) supplemented with 10 ng/ml bFGF, 100 U penicillin, 1000 U streptomycin, and 2 mM L-glutamine (Gibco BRL)). Cells were allowed to adhere overnight and non-adherent cells were washed out with medium changes. Medium changes were carried out twice weekly thereafter.

Maintenance and expansion: Once adherent cells reached approximately 50-60% confluency, they were detached with 0.25% trypsin-EDTA (Gibco BRL), washed twice with PBS (Gibco BRL), centrifuged at 1000 rpm for 5 minutes, and then replated at 1:3 under the same culture conditions.

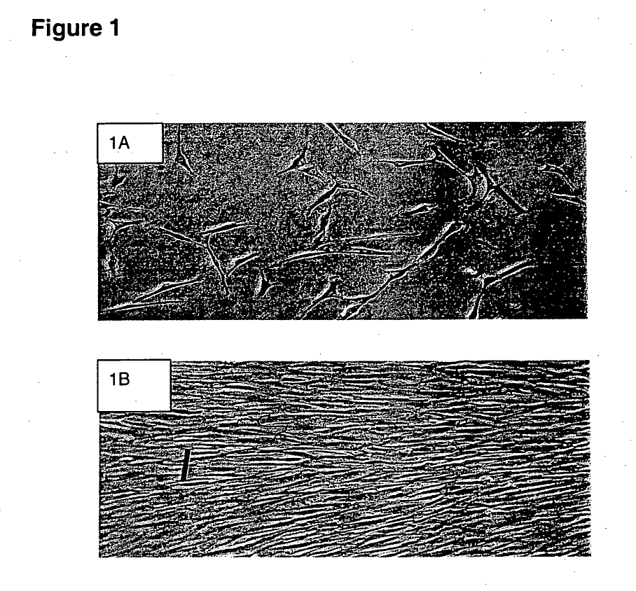

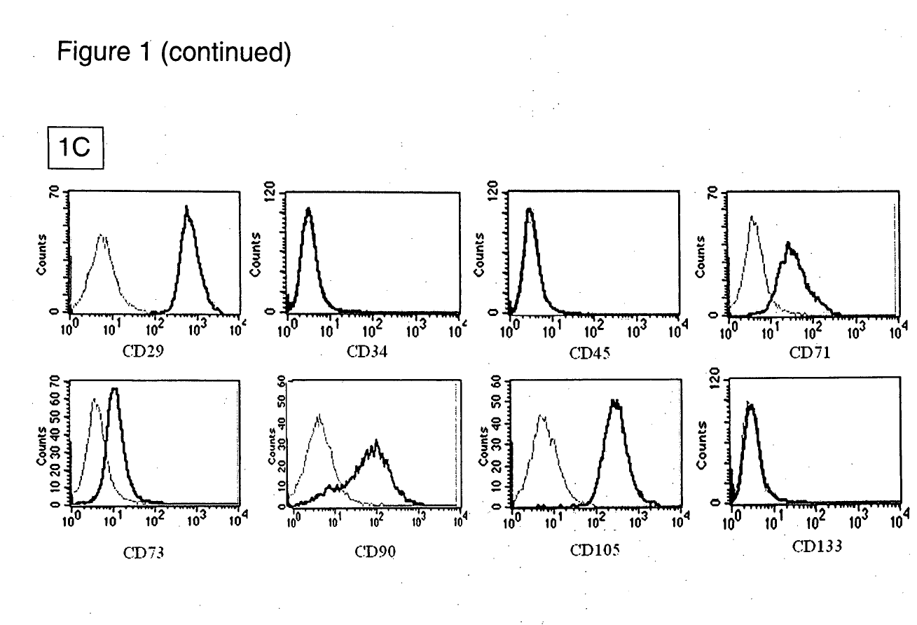

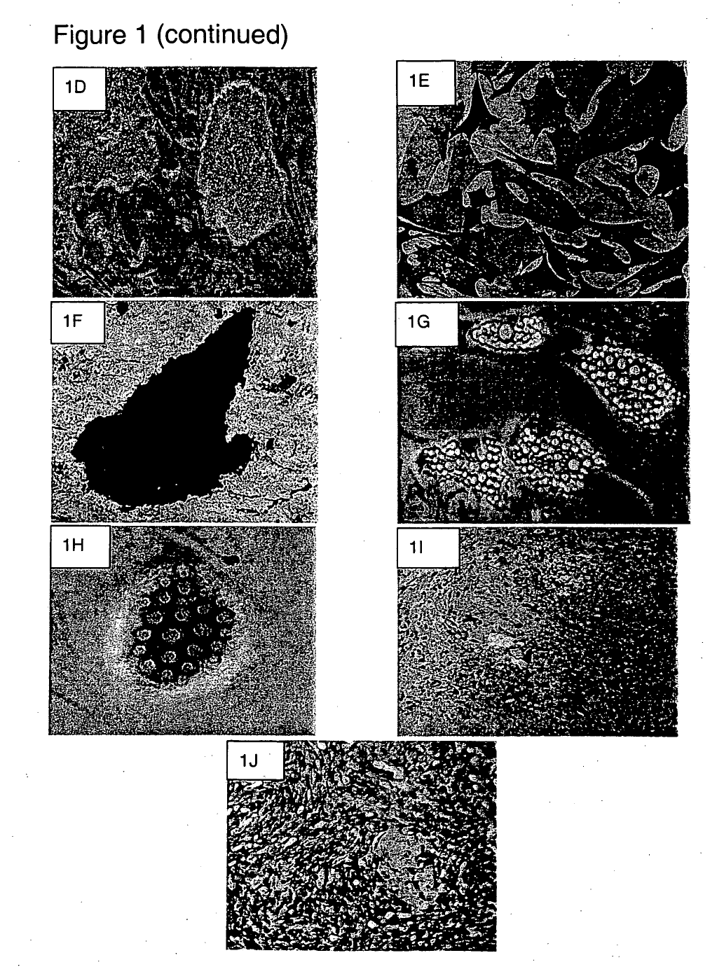

The fibroblast-like morphology of MSCs, at low density (FIG. 1A) and high confluence after expansion (FIG. 1B), and their immunophenotypic characterization (FIG. 1C), as determined by flow cytometry, are all consistent with that reported in the literature for bone marrow-derived MSCs (Devine, Cell Biochem Suppl 2002, 38:73-79). These cells were negative for CD34, CD45, and CD133 (AC133), but positive for CD29 (β1-integrin), CD71 (transferrin receptor), CD73, CD90 (Thy-1), and CD105 (endoglin).

### EXAMPLE 2

### In Vitro Differentiation

Osteogenic differentiation: To induce osteogenic differentiation, fifth- to seventh-passage cells were treated with osteogenic medium (IMDM supplemented with 0.1 μM dexamethasone (Sigma), 10 mM β-glycerol phosphate (Sigma), and 0.2 mM ascorbic acid (AsA, Sigma)) for three weeks with medium changes twice weekly. Osteogenesis was assessed at weekly intervals. The resulting osteocyte-like cells (FIG. 1D) showed positive alkaline phosphatase (FIG. 1E) and von kossa stainings (FIG. 1F).

Chondrogenic differentiation: To induce chondrogenic differentiation, fifth- to seventh-passage cells were transferred into 15 ml polypropylene: tubes and centrifuged at 1000 rpm for 5 minutes to form a pelleted micromass at the bottom of the tube. The cells were then treated with chondrogenic medium (high-glucose Dulbecco's-modified Eagle's medium (DMEM) (Bio-fluid, Rockville, Md., USA) supplemented with 0.1 μM dexamethasone, 50 μg/ml AsA, 100 μg/ml sodium pyruvate (Sigma), 40 μg/ml proline (Sigma), 10 ng/ml TGF-β1, and 50 mg/ml ITS⁺ premix (Becton Dickinson, 6.25 ug/ml Insulin, 6.25 ug/ml Transferrin, 6.25 ng/ml Selenious acid, 1.25 mg/ml BSA, and 5.35 mg/ml linoleic acid)) for three weeks. Medium changes were carried out twice weekly and chondrogenesis was assessed at weekly intervals. These chondrocyte-like cells show positive Safranin-O stain (FIG. 1I) and type II collagen expression (FIG. 1J), which are phenotypic markers of differentiated chondrocytes.

Adipogenic differentiation: To induce adipogenic differentiation, fifth- to seventh-passage cells were treated with adipogenic medium (IMDM supplemented with 0.5 mM 3-isobutyl-1-methylxanthine (IBMX, Sigma), 1 μM hydrocortisone (Sigma), 0.1 mM indomethacin (Sigma), and 10% rabbit serum (Sigma)) for three weeks. Medium changes were carried out twice weekly and adipogenesis was assessed at weekly intervals. These adipocyte-like cells (FIG. 1G) stained positive for Oil Red-O (FIG. 1H), a phenotypic marker of differentiated adipocytes.

### EXAMPLE 3

### In Vitro Hepatogenic Differentiation

To induce hepatogenic differentiation, fifth- to seventh-passage cells, at approximately 60% confluence, were serum-deprived for 2 days to synchronize cells in IMDM supplemented with 20 ng/ml EGF and 10 ng/ml FGF-2, followed by hepatic induction with differentiation medium containing IMDM supplemented with 20 ng/ml HGF, 10 ng/ml FGF-2, and 0.61 g/L nicotinamide for 7 days. The differentiation medium was then removed and replaced with hepatic maturation medium containing IMDM supplemented with 20 ng/ml OSM, 1 μM dexamethasone, and 1% (v/v) 50 mg/ml ITS⁺ premix. Medium changes were carried out twice weekly and hepatogenesis was assessed by RT-PCR at the time points indicated.

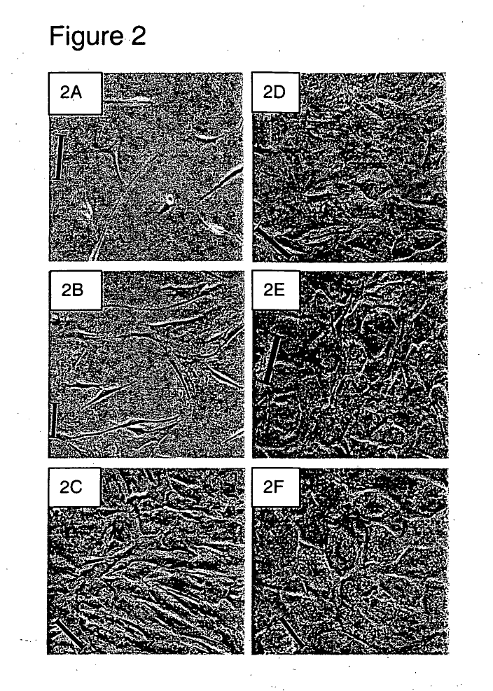

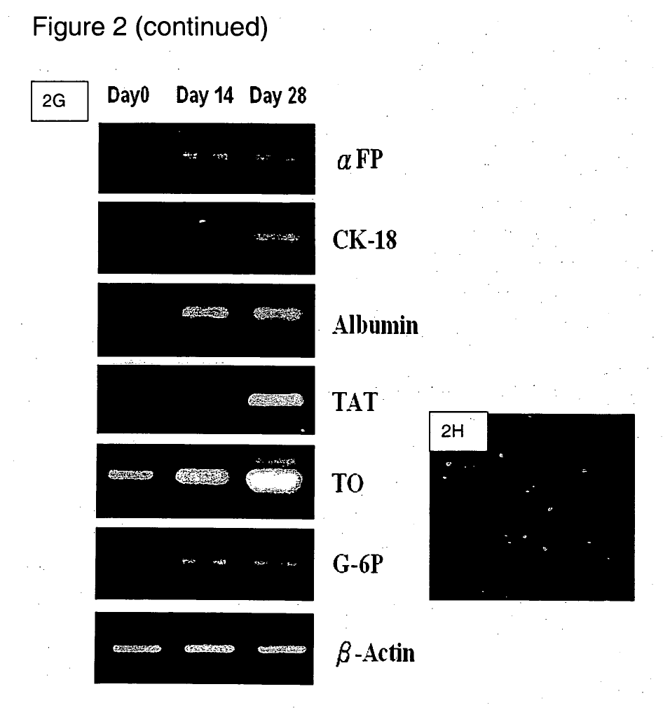

In the presence of HGF and FGF, the fibroblastic morphology (FIG. 2A) of the marrow-derived MSCs was lost and the cells developed a broadened, irregular morphology 1 week post induction (FIG. 2B). In the presence of OSM and ITS⁺, a retraction of elongated ends was observed 2 weeks post induction (FIG. 2C), and the cuboidal morphology of hepatocytes was visualized by 4 weeks post induction (FIG. 2D). The cuboidal morphology further matured with the appearance of abundant granules in cytoplasm after prolonged culture in the presence of OSM and ITS′ (FIG. 2E), and was retained for over 12 weeks (FIG. 2F).

### EXAMPLE 4

### Histological, Cytochemical and Immunocytochemical Analysis

Antibodies: Antibodies against human antigens CD29, CD34, CD45, CD71, CD73, CD90, and CD105 were purchased from Becton Dickinson. Antibodies against human antigen Cb133 were purchased from Miltenyi Biotec (Bergisch Gladbach, Germany). Antibodies against human albumin and secondary goat anti-mouse antibodies were from Dako (Carpinteria, Calif.). Monoclonal antibody against human 9B2 was a kind gift from Dr. C. P. Hu, Veterans General Hospital—Taipei, Taiwan (Chiu et al., Hepatology 1990, 11:834-842).

Cytochemical staining: For evaluation of mineralized matrix in osteogenic differentiated cells, cells were fixed with 4% formaldehyde and stained with 1% Alizarin-red S (Sigma) solution in water for 10 minutes. In addition, mineralized matrix was also evaluated by Von Kossa staining using 1% silver nitrate (Sigma) under ultra-violet light for 45 minutes, followed by 3% sodium thiosulphate (Sigma) for 5 minutes, and then counterstained with Van Gieson (Sigma) for 5 minutes. For Oil-red O staining, cells were fixed with 4% formaldehyde, stained with Oil-red-O (Sigma) for 10 minutes, and then counterstained with Mayers haematoxylin (Sigma) for 1 minute.

Histological analysis: Chondrogenic differentiation was evaluated after pellets were fixed in 4% formaldehyde, dehydrated in serial ethanol dilutions, and embedded in paraffin blocks. Blocks were cut and sections stained with Safranin-O (Sigma).

Immunofluorescence: For staining of intracellular proteins, cells were fixed overnight with 4% formaldehyde, at 4° C., and permeabilized with 0.1% Triton X-100 (Sigma) for 10 minutes. Slides and dishes were incubated with mouse primary antibodies against human albumin (1:50) for 1 hour, followed by incubation with fluorescein- or phycoerythrin-coupled goat anti-mouse IgG secondary antibody for 1 hour. Between incubations, slides and dishes were washed with PBS. Undifferentiated cells were negative for albumin by immunofluorescence analysis (not shown) while differentiated cells were strongly positive (FIG. 2H), indicating that the MSC-derived hepatocytes are expressing albumin, a liver-specific protein.

Flow cytometry: For cell surface antigen phenotyping, fifth- to seventh-passage cells were detached and stained with fluorescein- or phycoerythrin-coupled antibodies and analyzed with FACSCalibur® (Becton Dickinson).

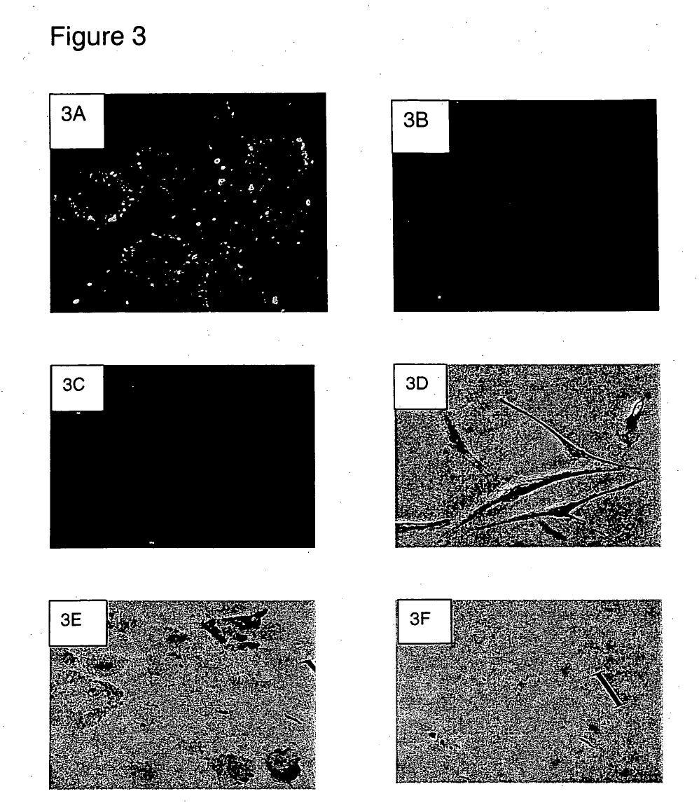

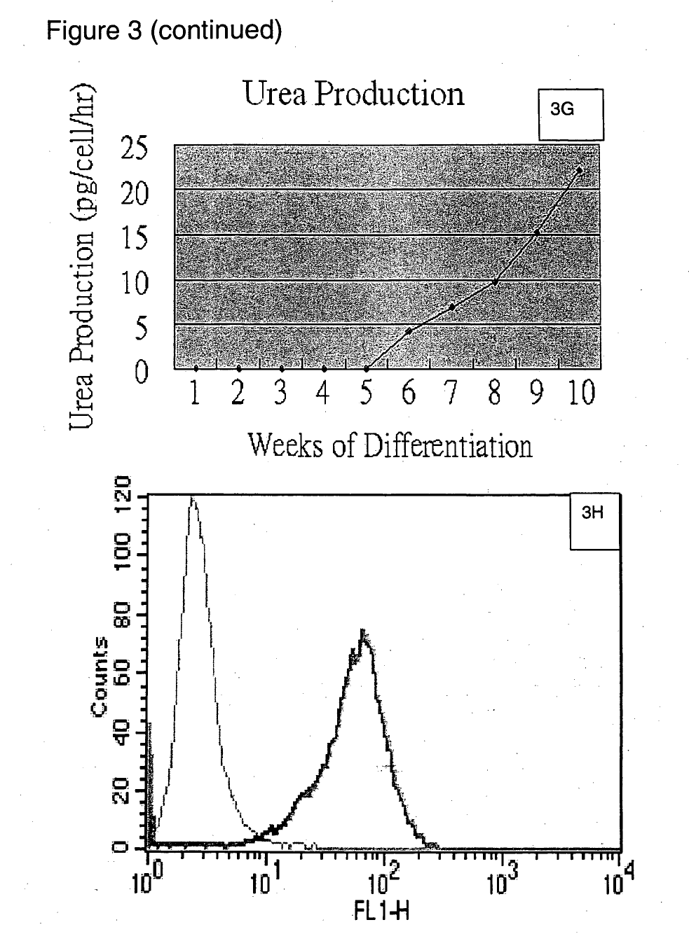

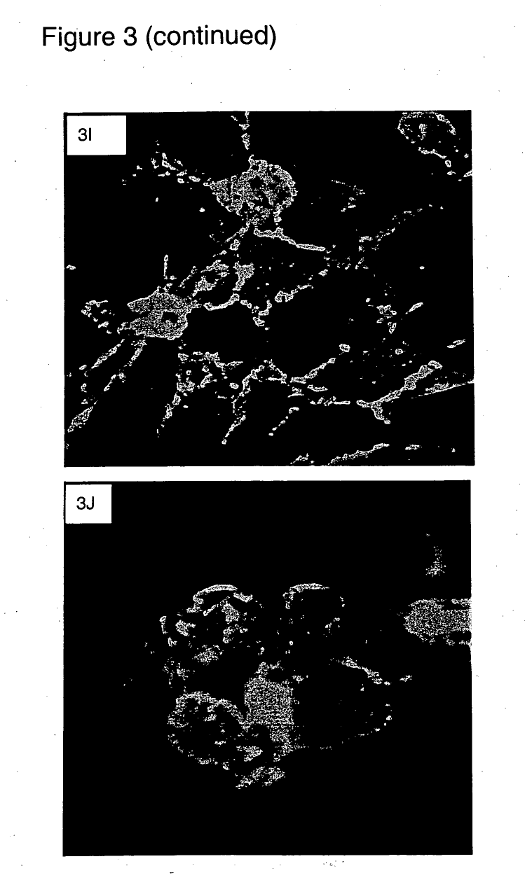

The monoclonal antibody 9B2 is a liver-specific antibody found to react with an antigen expressed on the bile canaliculi formed between adjacent hepatocytes (Chiu et al., Hepatology 1990, 11:834-842). Analysis by flow cytometry (FIG. 3H) revealed that differentiated hepatocyte-like cells were positive for the expression of antigen 9B2, and immunofluorescence assays further showed that the antibody was predominantly localized on the surface membrane bordering adjacent differentiated cells (FIG. 3I). Immunofluorescence analysis on hepatoma cell line Hep3B show a similar staining pattern (FIG. 3J). These results indicate that the MSC-derived hepatocytes are expressing liver-specific antigens on their cell surface.

### EXAMPLE 5

### Total RNA Isolation and RT-PCR

RNA was extracted from 3-30×10⁵ undifferentiated MSCs, partially differentiated MSCs, or differentiated MSC-derived hepatocytes using RNEasy® (Qiagen, Stanford, Valencia, Calif.). The mRNA was reverse transcribed to cDNA using Advantage RT-for-PCR® (Clontech, Palo Alto, Calif.). cDNA was amplified using a ABI GeneAmp® PCR System 2400 (Perkin Elmer Applied Biosystems, Boston, Mass.) at 94° C. for 40 seconds, 56° C. for 50 seconds, and 72° C. for 60 seconds for 35 cycles, after initial denaturation at 94° C. for 5 minutes. Primers used for amplification are listed in Table 1.

TABLE 1

| | | |
|---|---|---|
| Primer | Sequence | Product |

| | | |
|---|---|---|
| α-FP | S: 5′-TGCAGCCAAAGTGAAGAGGGAAGA-3′ | 216 bp |
| (α-fetoprotein) | (SEQ ID NO:1) | |
| | A: 5′-CATAGCGAGCAGCCCAAAGAAGAA-3′ | |
| | (SEQ ID NO:2) | |
| Albumin | S: 5′-TGCTTGAATGTGCTGATGACAGGG-3′ | 161 bp |
| | (SEQ ID NO:3) | |
| | A: 5′-AAGGCAAGTCAGCAGGCATCTCATC-3′ | |
| | (SEQ ID NO:4) | |
| CK-18 | S: 5′-TGGTACTCTCCTCAATCTGCTG-3′ | 148 bp |
| (Cytokeratin 18) | (SEQ ID NO:5) | |
| | A: 5′-CTCTGGATTGACTGTGGAAGT-3′ | |
| | (SEQ ID NO:6) | |
| TAT | S: 5′-TGAGCAGTCTGTCCACTGCCT-3′ | 358 bp |
| (Tyrosine- | (SEQ ID NO:7) | |
| aminotransferase) | A: 5′-ATGTGAATGAGGAGGATCTGAG-3′ | |
| | (SEQ ID NO:8) | |
| TO | S: 5′-ATACAGAGACTTCAGGGAGC-3′ | 299 bp |
| (Tryptophan 2,3- | (SEQ ID NO:9) | |
| dioxygenase) | A: 5′-TGGTTGGGTTCATCTTCGGTATC-3′ | |
| | (SEQ ID NO:10) | |
| G-6-P (Glucose- | S: 5′-GCTGGAGTCCTGTCAGGCATTGC-3′ | 350 bp |
| 6-phosphatase) | (SEQ ID NO:11) | |
| | A: 5′-TAGAGCTGAGGCGGAATGGGAG-3′ | |
| | (SEQ ID NO:12) | |
| β-actin | S: 5′-TGAACTGGCTGACTGCTGTG-3′ | 174 bp |
| | (SEQ ID NO:13) | |
| | A: 5′-CATCCTTGGCCTCAGCATAG-3′ | |
| | (SEQ ID NO:14) | |

RT-PCR analysis (FIG. 2G) showed that expression of α-fetoprotein (αFP) and glucose 6-phosphatase (G6P) were detectable by day 14, while tyrosine-aminotransferase (TAT), a late marker gene of hepatocytes (Hamazaki et al., FEBS Lett 2002, 497:15-19), was detected by day 28. Expression of cytokeratin-18 (CK-18), albumin, and tryptophan 2,3-dioxygenase (TO) were detected at all time points and increased with time of differentiation. Undifferentiated cells did not express αFP, TAT, or G6P, but did express low levels of albumin and CK-18, and TO, indicating that the MSC-derived hepatocytes are expressing increased levels of liver-specific genes.

### EXAMPLE 6

### Periodic Acid-Schiff for Glycogen

Petri dishes containing cells were fixed in 4% formaldehyde, permeabilized with 0.1% Triton X-100 for 10 minutes and were either not incubated or incubated with Diastase for one hour at 37° C. Samples were then oxidized in 1% periodic acid for 5 minutes, rinsed 3 times in dH₂O, treated with Schiff's reagent for 15 minutes, and then rinsed in dH₂O for 5-10 minutes. Samples were counterstained with Mayer's hematoxylin for 1 minute and rinsed in dH₂O and assessed under light microscope.

Undifferentiated cells stained negative (FIG. 3D) for glycogen, as determined by Periodic acid-Schiff (PAS) assay, while differentiated cells stained positive (FIG. 3E). When pre-treated with Diastase to digest glycogen, differentiated cells became negative for PAS staining (FIG. 3F). The afore-tested functions were sustained for over 12 weeks in these MSC-derived hepatocytes (not shown).

### EXAMPLE 7

### Uptake of Low-Density-LiPoprotein

The Dil-Ac-LDL staining kit was purchased from Biomedical Technologies (Stoughton, Mass.) and the assay was performed per manufacturer's instructions. After 6 weeks of differentiation the MSC-derived hepatocytes demonstrated the ability to take up LDL (FIG. 3A), while undifferentiated cells failed to take up LDL (not shown). The LDL uptake was sustained for over 12 weeks in the MSC-derived hepatocytes (not shown). LDL uptake is a characteristic of liver cells. Accordingly, the MSC-derived hepatocytes demonstrate liver-specific activities for up to 12 weeks.

### EXAMPLE 8

### Pentoxyresorufin Assay

After 6 weeks of differentiation, cells were maintained under the same conditions in the presence and absence of 1 mM phenobarbital and then incubated in the presence of pentoxyresorufin (PROD) for overnight and assessed under fluorescence microscope. Pentoxyresorufin is a non-fluorescent compound O-dealkylatable by cytochrome P450 (mainly CYP 2B and 2F isofamilies (Verschoyle et al., Xenobiotica 1997, 27:853-64)) into resorufin, emitting a red fluorescence. In the absence of phenobarbital, pentoxyresorufin was metabolized and red fluorescence was visualized in the MSC-derived hepatocytes (FIG. 3B), suggesting the existence of endogenous P450 enzymes in differentiated cells. Fluorescence was not observed in undifferentiated cells (not shown). In the presence of phenobarbital, an increase in fluorescence activity was observed (FIG. 3C), while undifferentiated cells remained non-fluorescent (not shown). The afore-tested functions were sustained for over 12 weeks in the MSC-derived hepatocytes (not shown). The existence of endogenous P450 enzymes is another indication that the MSC-derived hepatocytes are capable of functioning like liver cells.

### EXAMPLE 9

### Re-Expansion of MSC-Derived Hepatocytes

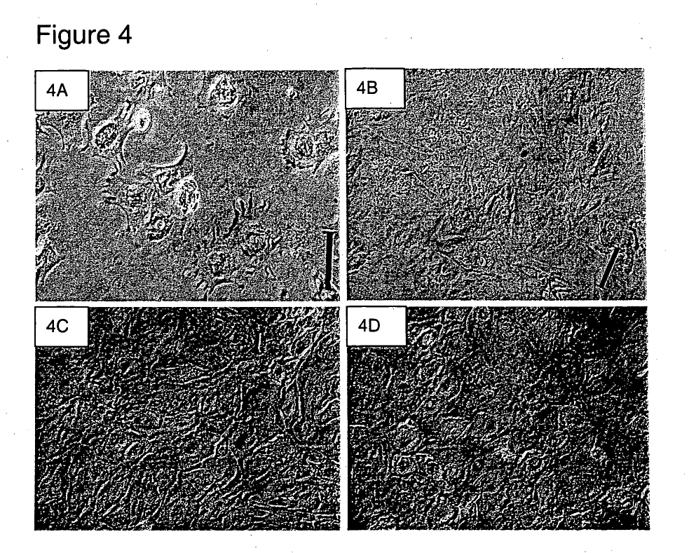

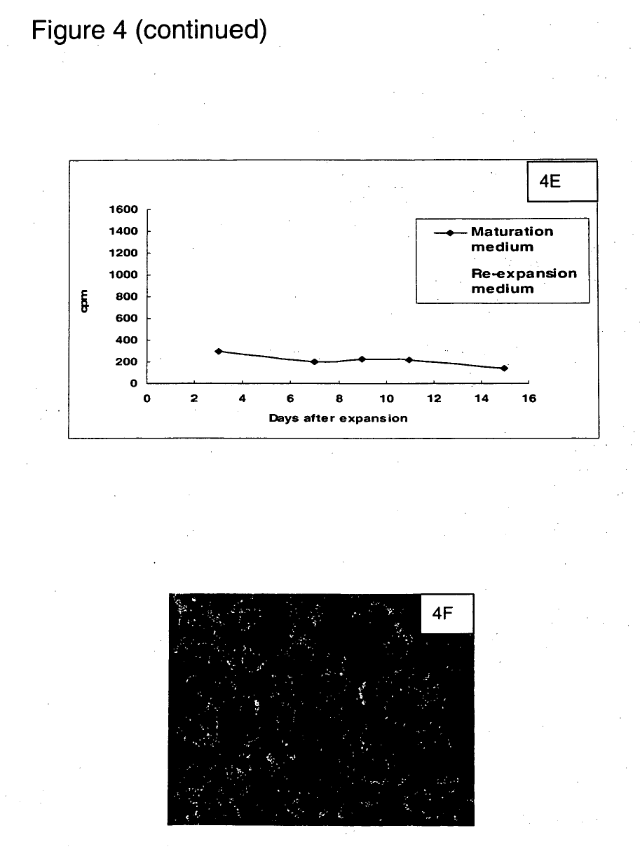

After maintaining MSC-derived hepatocytes under differentiating conditions for 6 weeks, the cells were passaged at 1:2 and cultured in IMDM supplemented with 10% FBS, 10 ng/ml HGF, 20 ng/ml OSM, 1 μM dexamethasone, and 50 mg/ml ITS⁺ premix for expansion, and then cultured in serum-free maturation medium once 80-90% confluence was reached. After 6 weeks of induction the MSC-derived hepatocytes oh a confluent dish were split at 1:4 (FIG. 4A) and, in the presence of re-expansion medium containing FBS, HGF, and OSM, the cuboidal morphology of hepatocytes was lost while cells began to proliferate (FIG. 4B). The cuboidal morphology was re-established (FIG. 4C) once cultures reach 80-90% confluence, and further matured in the presence of maturation medium containing OSM (FIG. 4D). Proliferation of the MSC-derived hepatocytes in re-expansion medium was confirmed by tritium-thymidine (³H) incorporation analysis (FIG. 4E). Furthermore, the expanded cells retained in vitro functions characteristic of hepatocytes as shown by the ability to produce albumin (not shown) and take up low-density lipoproteins (FIG. 4F), suggesting that the MSC-derived hepatocytes can be re-expanded to a large scale.

The examples described above were also performed successfully with MSC-derived hepatocytes incubated for 5 to 21 days in a first incubation media lacking FGF and/or nicotinamide, and then incubated for 2 to 12 weeks with a second incubation media lacking insulin and/or dexamethasone.

The specification is most thoroughly understood in light of the teachings of the references cited within the specification, which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents cited and sequences identified by accession or database reference numbers in this disclosure are incorporated by reference in their entirety. To the extent the material incorporated by reference contradicts or is inconsistent with the present specification, the present specification will supercede any such material. The citation of any references herein is as not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for inducing differentiation of mesenchymal stem cells into hepatocytes in vitro comprising incubating cultured mesenchymal stem cells with a first culture media comprising hepatocyte growth factor followed by incubating the cells with a second culture media comprising oncostatin-M.

2. The method of claim 1, wherein the mesenchymal stem cells comprise cells isolated from the iliac crest of a patient donor.

3. The method of claim 1, wherein the mesenchymal stems cells are subjected to immunodepletion of cells expressing CD3, CD14, CD19, CD38, CD66b, and/or glycophorin A before culturing.

4. The method of claim 1, wherein the mesenchymal stem cells are of human origin.

5. The method of claim 1, wherein the first culture media further comprises fibroblast growth factor, nicotinamide, or both.

6. The method of claim 1, wherein the second culture media further comprises dexamethasone, insulin, or both.

7. A composition comprising mesenchymal stem cell (MSC)-derived hepatocytes.

8. The composition of claim 7, wherein the MSC-derived hepatocytes are produced by the method of claim 1.

9. The composition of claim 7, wherein the mesenchymal stem cells are isolated from the iliac crest of a patient donor.

10. The composition of claim 7, wherein the mesenchymal stems cells are subjected to immunodepletion of cells expressing CD3, CD14, CD19, CD38, CD66b, and/or glycophorin A before culturing.

11. The composition of claim 7, wherein the mesenchymal stem cells are of human origin.

12. The composition of claim 7, wherein the first culture media further comprises fibroblast growth factor, nicotinamide, or both.

13. The composition of claim 7, wherein the second culture media further comprises dexamethasone, insulin, or both.

14. A method for repairing liver damage in a patient, comprising administering MSC-derived hepatocytes.

15. The method of claim 14, wherein the MSC-derived hepatocytes are produced by the method of claim 1.

16. A method for growing liver tissue in vitro comprising repeated culturing and expansion of MSC-derived hepatocytes.

17. The method of claim 16, wherein the culturing and expansion occurs in a culture media comprising hepatocyte growth factor, oncostatin-M, or both.

18. A composition comprising liver tissue produced by the method of claim 16.

19. A method for the in vitro growth of liver-specific viruses comprising incubating the virus with a composition of claim 7 or 18.

20. A method of screening a compound for its effect on hepatocytes or hepatocyte activity, comprising:
a) combining the compound with a composition of claim 7 or 18;
b) determining any change to MSC-derived hepatocytes or their activity as a result of contact with the compound; and
c) correlating the change with the effect of the compound on hepatocytes or hepatocyte activity.

21. The method of claim 20, further comprising determining whether the compound is toxic to MSC-derived hepatocytes.

22. The method of claim 20, further comprising determining whether the compound affects ability of MSC-derived hepatocytes to proliferate or be maintained in culture.

23. The method of claim 20, further comprising determining whether the compound changes enzyme activity or secretion normally present in MSC-derived hepatocytes.

24. The method of claim 20, wherein the MSC-derived hepatocytes have been genetically altered.

25. A kit for the preparation of MSC-derived hepatocytes comprising a first culture media comprising hepatocyte growth factor, and a second culture media comprising oncostatin-M.

26. The kit of claim 25, wherein the first culture media further comprises FGF, nicotinamide, or both.

27. The kit of claim 25, wherein the second culture media further comprises dexamethasone, insulin, or both.

28. The kit of claim 25 further comprising cultured mesenchymal stem cells.

29. A kit for the in vitro growth of liver-specific viruses comprising MSC-derived hepatocytes.
